(19) United States
(12) Patent Application Publication
(10) Pub. No.: US 2022/0135750 A1
(43) Pub. Date: May 5, 2022

(54) SURGICAL HYDROGEL

(71) Applicant: CHITOGEL LIMITED, Dunedin (NZ)

(72) Inventors: Simon Rae Robinson, Wellington (NZ); Smita Ghosh, Lower Hutt (NZ)

(21) Appl. No.: 17/429,045

(22) PCT Filed: Jan. 31, 2020
(86) PCT No.: PCT/NZ2020/050004

§ 371 Date: Aug. 6, 2021

Related U.S. Application Data
(60) Provisional application No. 62/803,119, filed on Feb. 8, 2019.

(51) Int. Cl. C08J 3/075; A61L 26/00; A61L 31/04; A61L 31/14

(52) U.S. Cl. CPC C08J 3/075; A61L 26/008; A61L 26/0052; C08J 2305/02; A61L 31/145; A61L 2400/04; C08J 2305/08; A61L 31/041

(57) ABSTRACT

Disclosed are surgical hydrogels derived from the combination of chitosan derivative and aldehyde-derivatised dextran polymers in combination with a humectant for use as surgical wound packing materials or stents. Also disclosed are sterile kits comprising the precursor components of the surgical hydrogels. Also disclosed are methods of sterilizing the kits and individual components thereof for preparing the hydrogels.

### FIELD

The present invention relates to surgical hydrogels for use as surgical wound packing material or stent, sterile kits for preparing the surgical hydrogels, and methods for sterilising the kits for preparing surgical hydrogels.

### BACKGROUND

Any discussion of the prior art throughout the specification should in no way be considered as an admission that such prior art is widely known or forms part of common general knowledge in the field.

Endoscopic sinus surgery (ESS) is one of the most common surgical procedures globally with approximately 500,000 cases performed annually in the United States alone. For example, ESS is widely used as an effective treatment for chronic sinusitis.

Post-operative complications of ESS include excessive bleeding, adhesions, granulation, edema and other problems such as ostial stenosis, or the reduction in ostial patency, and infections.

Traditionally, removable packing had been used for stopping excessive bleeding and reducing adhesions. However, the removal of this packing can be very painful for the patients and is associated with significant mucosal trauma.

In recent years there has been increased interest in the use of hydrogels as packing materials. Hydrogels are a hydrophilic polymeric network and have many biological applications due to their soft flexible nature and high-water content.

To be effective in the treatment of surgical wounds, especially surgical wounds associated with ESS, the hydrogel requires a particular consistency, or viscosity. The hydrogel must be able to be applied via a syringe or through a tube to the site of the wound, adhere to the site of the wound and remain in place during at least the initial stages of healing.

A hydrogel packing material of particular promise as a wound packing material for surgical wounds comprises a polymer network comprising a chitosan derivative cross-linked with an aldehyde derivatised dextran polymer. The hydrogel comprising a chitosan derivative cross-linked with an aldehyde derivatised dextran polymer is described in WO2009/028965, the contents of which are fully incorporated by reference.

One challenge with the chitosan derivative/aldehyde derivatised dextran polymer hydrogel as a wound packing material is ensuring that the hydrogel has the appropriate consistency or viscosity.

The inventors of the present invention have thus devised a kit comprising stable and sterile precursor materials for the chitosan derivative/aldehyde derivatised dextran hydrogel which has a commercially viable shelf life and can be easily used by health care workers to prepare the surgical hydrogel, for example, during or shortly before a surgical procedure in which the hydrogel will be used.

A challenge with providing a kit for use in surgical procedures is that the entirety of the kit must meet the rigorous sterility requirements of an operating theatre, or be sufficiently sterile to minimise or prevent the possibility of infections in the patient. The process of sterilization refers to any action used to eliminate or kill any form of life present on a surface or contained in a liquid. Effectively sterilising a surgical kit comprising hydrogel precursors requires the determination of a sterilisation strategy that effectively sterilises every component of the kit without degrading any one component.

It is accordingly an object of the present invention to provide a sterile kit for preparing a hydrogel stent, or wound packing material comprising sterile hydrogel precursor components, that have a commercially viable shelf life.

### SUMMARY OF THE INVENTION

It has been found that, over time, the consistency of chitosan derivative/aldehyde derivatised dextran hydrogel will vary. It has been found that the shelf life of the hydrogel (being the period of time in which the hydrogel has a stable consistency) is on the order of days or weeks, even in air-tight and sterile packaging. This appears to be due to variations in the degree of cross-linking between the chitosan derivative and aldehyde derivatised dextran. Thus, the shelf life of the hydrogel is so short that it is not commercially viable to manufacture and sell the hydrogel as a ready-to-use product.

The present invention is predicated, at least in part, on the discovery of inherent instability of aldehyde derivatised dextran polymers in aqueous solution, the discovery of the instability of hydrogels prepared from the cross linking of aldehyde derivatised dextran and chitosan derivative, and the discovery of the susceptibility of these components to known methods of sterilisation.

Accordingly, the present invention relates, at least in part, to the provision of a sterile kit for preparing the hydrogel comprising three parts (a solid aldehyde derivatised dextran component, a chitosan derivative solution, and a buffer solution) and a method for sterilising the kit, such that the kit has a commercially viable shelf life.

The inventions described and claimed herein have many attributes and embodiments including, but not limited to, those set forth or described or referenced in this Summary of the Invention. It is not intended to be all-inclusive and the inventions described and claimed herein are not limited to or by the features or examples identified in this Summary of the Invention, which is included for purposes of illustration only and not restriction.

The present invention provides a kit for preparing a medical hydrogel. The hydrogel is particularly useful as a surgical stent or wound packing material. As will be described in more detail below, the kit comprises separate sterile hydrogel precursors which are combined before use to prepare the hydrogel. The precursors are individually packaged, and the individual precursors may be combined in a kit. The kit may contain pre-measured, individually packaged, quantities of each precursor, the equipment necessary to combine each precursor, and instructions for use.

Production of the kit of the present invention has overcome challenges in ensuring that each component of the kit, and the entire kit itself, is sufficiently sterilised for use in an operating theatre. Particular challenges arise where components of the kit are susceptible to degradation under different sterilisation conditions. Whilst sterilisation methods for each individual hydrogel precursors may be known, determining a sterilisation protocol of the kit as a whole is complicated by the sensitivities of hydrogel precursors to degradation and the properties of the packaging used in the kit.

### Hydrogel

In one aspect, there is provided a hydrogel for use in post-operative care and surgical wound healing comprising a dicarboxy-derivatised chitosan polymer cross-linked to an aldehyde-derivatised dextran polymer in aqueous solution.

In an embodiment, the hydrogel further comprises a humectant. In a related embodiment, the humectant is glycerol.

In another embodiment, the dicarboxy-derivatised chitosan polymer is crosslinked to the aldehyde-derivatised dextran polymer through the amine group of the dicarboxy-derivatised chitosan polymer and the aldehyde group of the aldehyde-derivatised dextran polymer.

In an embodiment, the dicarboxy-derivatised chitosan polymer is an N-succinyl chitosan polymer (also known as chitosan N-succinamide polymer).

In an embodiment, the N-succinyl chitosan is cross-linked to an aldehyde-derivatised dextran polymer. In an embodiment, the N-succinyl chitosan is cross-linked to the aldehyde-derivatised dextran polymer through the amine group of the N-succinyl chitosan and the aldehyde group of the aldehyde-derivatised dextran polymer.

In an embodiment, the hydrogel comprises between about 2% to 10% w/v dicarboxy-derivatised chitosan polymer. In an embodiment, the hydrogel comprises between about 2% to 10% w/v aldehyde-derivatised dextran polymer.

Preferably, the hydrogel comprises between about 2% to about 8% w/v, more preferably between about 2% to about 6% w/v dicarboxy-derivatised chitosan polymer. Most preferably, the hydrogel comprises about 5% w/v dicarboxy-derivatised chitosan polymer.

Preferably, the hydrogel comprises between about 2% to about 8% w/v, more preferably between about 2% to about 6% w/v aldehyde-derivatised dextran polymer. Most preferably, the hydrogel comprises about 3% w/v aldehyde-derivatised dextran polymer.

In an embodiment, the ratio of aldehyde-derivatised dextran polymer to N-succinyl chitosan polymer is between 1:0.2 and 1:1, more preferably between 1:0.4 and 1:0.8, most preferably 1:0.6.

In an embodiment, the water content of the hydrogel is between about 60% and about 80%, more preferably between about 65% and about 75%, most preferably about 72%.

In an embodiment, the hydrogel comprises between 10% and about 30% w/v of a humectant, more preferably between about 15% and about 25%, most preferably 20%. In an embodiment, the humectant is glycerol.

In an embodiment, the water content of the hydrogel at least partially comprises a buffer solution. Preferred buffer solutions include sodium phosphate buffer solutions. Preferred concentrations include 0.3% phosphate buffer. The buffer solution preferably has a pH of between about 7 and 8, more preferably between about 7.2 and 7.6.

In an embodiment the aqueous solution of aldehyde-derivatised dextran polymer has a pH of between about 6 and 8. Preferably, the aqueous solution of aldehyde-derivatised dextran polymer has a pH of between about 6.5 and 7.5.

### Kit

In a further aspect, there is provided a kit for preparing the hydrogel of the invention, comprising the following hydrogel precursors:
A dicarboxy-derivatised chitosan polymer
An aldehyde-derivatised dextran polymer

In an embodiment the dicarboxy-derivatised chitosan polymer is an N-succinyl chitosan polymer.

In an embodiment, the dicarboxy-derivatised chitosan polymer, such as N-succinyl chitosan, is provided as a solution. In this example, the derivatised chitosan polymer may be suspended in water, saline, or a buffer solution, such as a sodium phosphate buffer. In an embodiment, the aqueous solution of dicarboxy-derivatised chitosan polymer has a pH between about 6.5 and 7.5. In an embodiment, the concentration of the N-succinyl chitosan polymer in solution is between about 1% w/v and about 10% w/v. In an embodiment, the concentration is between about 3% w/v and about 7% w/v, most preferably about 5% w/v.

In an embodiment, the aldehyde-derivatised dextran polymer is provided as a dry powder. Provision of the aldehyde-derivatised dextran polymer component as a dry powder improves the stability of the component, and thus extends the shelf life of the kit.

The kit may further include a buffer solution, such as a sodium phosphate buffer solution for suspending the aldehyde-derivatised dextran polymer during the preparation of the hydrogel.

In an embodiment, the N-succinyl chitosan polymer is packaged and sealed in a first container. The first container may be a glass container such as a vial or ampoule. Where the first container has a lid, the lid may be made from plastic resin, metal or other suitable material.

In an embodiment, the aldehyde-derivatised dextran polymer is packaged and sealed in a second container. The second container may be a glass container such as a vial or ampoule. Where the second container has a lid, the lid may be made from plastic resin, metal or other suitable material.

In an embodiment, the kit further comprises a buffer solution. In an embodiment, the buffer solution is for suspending or dissolving the solid aldehyde-derivatised dextran polymer. In an embodiment, the buffer solution is for suspending or dissolving the dicarboxy-derivatised chitosan polymer. Any pharmaceutically acceptable buffer solution that is suitable for dissolving aldehyde-derivatised dextran polymer or dicarboxy-derivatised chitosan polymer may be used. The buffer solution preferably has a pH of between about 7 and 8, more preferably between about 7.2 and 7.6. In an embodiment, the concentration of the buffer solution is about 3% w/v. In an embodiment, the buffer is sodium phosphate. The buffer solution is packaged and sealed in a third container. The third container may be a glass container such as a vial or ampoule. Where the third container has a lid, the lid may be made from plastic resin, metal or other suitable material.

In an embodiment, the hydrogel further comprises a humectant. In an embodiment, the humectant is glycerol. The humectant may be provided as a separate component in the kit, or may alternatively be combined in a buffer solution. Alternatively, or in addition, the humectant may be combined in the dicarboxy-derivatised chitosan polymer solution. In an embodiment, the humectant is glycerol.

The amount of humectant provided in the kit corresponds to a quantity in the resulting hydrogel prepared from the kit of 10% and about 30% w/v, more preferably between about 15% and about 25% w/v, most preferably 20% w/v.

In an embodiment, the humectant is combined in a buffer solution. In an embodiment, there is provided a buffer solution comprising between about 30% w/v and about 50% w/v glycerol. In an embodiment, the glycerol content is 40% w/v. The buffer solution may be a sodium phosphate buffer solution.

The kit may further comprise equipment for mixing and preparing the hydrogel and equipment for applying the hydrogel to a surgical wound:
Fluid dispensing connector
Two (2) or more mixing cannulas
Pliable cannula
Two (2) 20 ml syringes

The pliable cannula of the kit has been developed to be advantageously used in conjunction with the hydrogel prepared from the kit of the present invention. In particular, the pliable cannula has been developed to apply the hydrogel of the present invention to surgical wounds associated with ESS procedures, such as surgical wounds in the sinus and/or the nasal cavity.

In an embodiment, the pliable cannula comprises a soft plastic tube and one or more pliable wires embedded in the plastic tube, wherein the pliable wire(s) extends partially or completely along the length of the plastic tube. In an embodiment, the pliability of the pliable cannula is derived from two pliable wires embedded in the plastic tube. In an embodiment, a first open end of the pliable cannula is configured to be attached to a syringe, for example via a luer lock or luer slip mechanism. In an embodiment, a second open end of the pliable cannula is configured to apply the hydrogel to the site of the surgical wound.

The pliable wire embedded in the tube allows the pliable cannula to be manually bent and shaped into a particular configuration so as to allow the cannula to be inserted via the nose (for example) or other body orifice to the site of the surgical wound. The pliability of the cannula thus allows the precise application of the hydrogel to the site of the surgical wound without causing further trauma to the patient.

The plastic tube itself is a soft plastic material which is sufficiently soft that it does not cause trauma to the patient during insertion of the cannula or application of the hydrogel.

In an embodiment, the pliable wire material is stainless steel.

In an embodiment, the kit further comprises a barrier system for housing the components of the kit. For example, the barrier system is adapted to house the hydrogel precursor components and the equipment for preparing the hydrogel from the precursor components. The barrier system is permeable to ethylene oxide. Once sterilised by ethylene oxide, the barrier system maintains sterility of the contents of the system.

In an embodiment, the barrier system comprises a plastic. In an embodiment, the barrier system comprises nonwoven HDPE fibre, such as Tyvek® (Du Pont).

In an embodiment, the barrier system is loaded with the sterilised hydrogel precursor component containers and optionally the equipment (optionally pre-sterilised) for preparing the hydrogel, and the barrier system is subsequently sterilised by ethylene oxide sterilisation. Optionally, the precursor components and equipment may be loaded into a thermoformed tray or packaging to protect and present the components for use in the operating theatre, prior to loading into the barrier system.

In an embodiment, following loading of the kit components, the barrier system is sealed. In an embodiment, sterilisation of the sealed barrier system and its contents is performed by ethylene oxide sterilisation.

The barrier system is intended to function as a sterile barrier system. For example, the barrier system should provide a microbial barrier and allow aseptic presentation of the hydrogel precursor components and equipment for preparing the hydrogel at the point of use. In this embodiment, the barrier system is permeable to ethylene oxide, such that the barrier system, and the components sealed inside the barrier system, may be effectively sterilised by exposure to ethylene oxide.

The kit may further comprise a protective packaging enclosing the barrier system.

The protective packaging functions to prevent damage to the barrier system and its components until the point of use. In an embodiment, the protective packaging comprises a sealable clamshell container. In another embodiment, the protective packaging comprises cardboard packaging.

The kit may further comprise a foam inner portion (optionally comprising indentations for receiving each component and/or piece of equipment), product labelling and instructions for use.

In an embodiment, the kit is a single-use kit. The single-use kit comprises each precursor component of the hydrogel in a quantity and/or relative proportion sufficient to prepare a quantity of hydrogel for application to the site of a surgical wound. In particular, the single-use kit provides sufficient quantities of precursor to prepare sufficient hydrogel for application to surgical wounds associated with ESS.

In an embodiment, each precursor component of the hydrogel in the kit is pre-measured for a single use preparation of hydrogel. The single use relates to application of the hydrogel to the surgical wounds of a single surgical procedure, such that the hydrogel functions as a stent or packing material. In particular, the surgical procedure is ESS. The amount of hydrogel necessary for application to surgical wounds associated with ESS is about 15 to 25 ml.

For example, a single-use kit may comprise individually packaged and sealed containers comprising:
Container A containing 8-15 ml Sodium Phosphate buffer solution with 40% w/v Glycerol
Container B containing 230-440 mg Aldehyde-derivatised dextran polymer powder
Container C containing 8-15 ml N-succinyl chitosan polymer solution

In an embodiment, the components are provided in the correct relative proportions such that the hydrogel may be easily and conveniently prepared from the precursor components.

In an embodiment, the aldehyde-derivatised dextran polymer is provided in an amount that, when the entirety of each component provided in the kit is combined, a hydrogel is prepared having between about 2% to about 8% w/v aldehyde-derivatised dextran polymer, more preferably between about 2% to about 6% w/v aldehyde-derivatised dextran polymer, most preferably about 3% w/v aldehyde-derivatised dextran polymer.

In an embodiment, the N-succinyl chitosan polymer solution is provided in an amount that, when the entirety of each component provided in the kit is combined, a hydrogel is prepared having between about 2% to about 8% w/v dicarboxy-derivatised chitosan polymer, more preferably between about 2% to about 6% w/v dicarboxy-derivatised chitosan polymer, most preferably about 5% w/v dicarboxy-derivatised chitosan polymer.

In an embodiment, the ratio of aldehyde-derivatised dextran polymer to N-succinyl chitosan polymer is between 1:0.2 and 1:1, more preferably between 1:0.4 and 1:0.8, most preferably 1:0.6.

In an embodiment, the sterilised components, contained in their sterilised and sealed containers, are shelf stable (i.e. undergo no detectable degradation at room temperature) for at least 12 months, preferably for at least 18 months, and more preferably for at least 24 months.

Accordingly, in an aspect of the invention, there is provided a kit for preparing a sterile hydrogel comprising:
a sterile first container comprising a sterile aqueous solution comprising N-succinyl chitosan polymer;
a sterile second container comprising a sterile solid aldehyde-derivatised dextran polymer;
a sterile third container comprising a sterile aqueous buffer solution for suspending the aldehyde-derivatised dextran polymer during the preparation of the hydrogel; and
a sterile barrier system enclosing the first, second and third containers;
wherein a humectant is present in either or both of the first and third containers.

In an embodiment, the humectant comprises glycerol.

In an embodiment, the kit further comprises equipment for preparing the hydrogel, such as syringes, cannulae and syringe to syringe connectors. The equipment may include a pliable cannula comprising a soft plastic tube and one or more pliable wires embedded in the plastic tube, wherein the pliable wire(s) extends partially or completely along the length of the plastic tube. In an embodiment, the pliable cannula comprises a first open end configured to be attached to a syringe and a second open end of the pliable cannula configured to apply the hydrogel to the site of a surgical wound associated with endoscopic sinus surgery.

In an embodiment, the N-succinyl chitosan is suspended in water, saline, or a buffer solution, such as a sodium phosphate buffer.

In an embodiment, the aldehyde-derivatised dextran polymer is provided as a dry solid.

In an embodiment, the concentration of the N-succinyl chitosan polymer solution is between about 1% and about 10% w/v.

In an embodiment, the relative proportions of N-succinyl chitosan polymer to aldehyde-derivatised dextran polymer present in the kit is between 1:0.2 and 1:1.

In an embodiment, the second container comprises between about 230 and about 440 mg solid aldehyde derivatised dextran polymer.

In an embodiment, the concentration of the N-succinyl chitosan polymer solution is between about 1% and about 10% w/v. In this embodiment, the first container comprises between about 8 and about 15 ml of N-succinyl chitosan polymer solution.

In an embodiment, the third container comprises between about 8 and about 15 ml of a buffer solution. In this embodiment, the buffer solution comprises between about 30% w/v and about 50% w/v humectant.

### Method of Sterilisation

Sterilization can not only kill disease-causing microorganisms but also eliminates transmissible agents such as spores and bacteria. Sterilisation is essential for materials used in a surgical procedure, or to even be present in some locations of an operating theatre. The choice of the method for sterilisation depended on the type of material to be sterilised.

In one aspect, there is provided a method for sterilising a kit of the present invention, the method comprising the steps of:
sterilising a solution of N-succinyl chitosan polymer by steam sterilisation;
sealing the sterilised solution of N-succinyl chitosan polymer in a first container;
sterilising a solid aldehyde-derivatised dextran polymer by gamma irradiation sterilisation;
sealing the sterilised aldehyde-derivatised dextran polymer in a second container;
sterilising an aqueous solution comprising a buffer;
sealing the sterilised aqueous buffer solution in a third container; and
sterilising a housing comprising the first, second and third sealed containers, by ethylene oxide sterilisation; and
sealing the housing closed such that the first, second and third sealed containers are housed in a sterile environment.

In an embodiment, the concentration of the N-succinyl chitosan polymer in solution is between about 1% w/v and about 10% w/v. In an embodiment, the concentration is between about 3% w/v and about 7% w/v, most preferably about 5% w/v. In an embodiment, the sterilisation of the solution of N-succinyl chitosan polymer by steam sterilisation is performed using an autoclave.

In an embodiment, the steam sterilization procedure uses saturated steam. In an embodiment, the steam sterilization procedure is performed at a constant temperature of about 121° C. (250° F.). In an embodiment, the steam sterilization procedure is performed for a holding time of at least 15 minutes.

In an embodiment, the aldehyde-derivatised dextran polymer is a solid powder. In an embodiment, the solid aldehyde-derivatised dextran polymer is amorphous. In an embodiment, the sterilisation of the aldehyde-derivatised dextran polymer powder is performed by exposing the aldehyde-derivatised dextran polymer to a dose of at least 25 kGy gamma radiation. In an embodiment, the dose of gamma radiation is between about 25 kGy and about 35 kGy.

The N-succinyl chitosan polymer solution is packaged and sealed in a first container. The first container may be a glass container such as a vial or ampoule. Where the first container has a lid, the lid may be made from plastic resin, metal or other suitable material. In an embodiment, the exterior of the sealed first container is sterilised by ethylene oxide sterilisation.

In an embodiment, the ethylene oxide sterilisation procedure is performed at a temperature of between 30° C. and 60° C. In an embodiment, the ethylene oxide sterilisation procedure is performed at a relative humidity above 30 percent. In an embodiment, the ethylene oxide sterilisation procedure is performed at an ethylene oxide concentration between 200 and 800 mg/l in air, nitrogen, carbon dioxide or other inert gas or mixture of gases. In an embodiment, the ethylene oxide sterilisation procedure is performed for a duration of at least three hours.

The aldehyde-derivatised dextran polymer is packaged and sealed in a second container. The second container may be a glass container such as a vial or ampoule. Where the second container has a lid, the lid may be made from plastic resin, metal or other suitable material. In an embodiment, the exterior of the sealed second container is sterilised by ethylene oxide sterilisation. The temperature of the sterilisation procedure of the exterior of the container must be low enough to not degrade the aldehyde-derivatised dextran polymer inside.

The kit may further comprise a buffer solution. The buffer solution is for suspending or dissolving the solid aldehyde-derivatised dextran polymer. Any pharmaceutically acceptable buffer solution that is suitable for dissolving aldehyde-derivatised dextran polymer may be used. Preferred buffer solutions include sodium phosphate buffer solutions. In an embodiment, the buffer solution has a pH of between about 7 and 8, more preferably between about 7.2 and 7.6. In an embodiment, the concentration of the buffer solution is about 3% w/v. The buffer solution is packaged and sealed in a third container. The third container may be a glass container such as a vial or ampoule. Where the third container has a lid, the lid may be made from plastic resin, metal or other suitable material. In an embodiment, the exterior of the third container comprising the buffer solution is sterilised by steam sterilisation.

In an embodiment, the kit further includes equipment for preparing the hydrogel from the hydrogel precursor components. In an example, the kit comprises syringes or other apparatus for drawing up liquid hydrogel precursor components, cannulae, syringe to syringe connectors and other apparatus for mixing the hydrogel precursors and preparing the hydrogel. In an embodiment, the equipment is sterilised by ethylene oxide sterilisation.

Accordingly, in an aspect of the invention, there is provided a method for sterilising a kit for preparing a sterile hydrogel, the method comprising the steps of:
i. sealing a solution of N-succinyl chitosan polymer in a first container;
ii. sterilising the sealed first container comprising the N-succinyl chitosan polymer by steam sterilisation;
iii. sealing a solid aldehyde-derivatised dextran polymer in a second container;
iv. sterilising the second container comprising the solid aldehyde-derivatised dextran polymer by gamma irradiation sterilisation;
v. sealing an aqueous buffer solution in a third container;
vi. sterilising the third container comprising the aqueous solution;
vii. sealing the first, second and third sterile sealed containers in a housing; and
viii. sterilising the housing comprising the first, second and third containers by exposure to ethylene oxide.

In an embodiment, the aldehyde-derivatised dextran polymer is a solid powder.

In an embodiment, the sterile aqueous solution comprising N-succinyl chitosan polymer, the sterile solid aldehyde-derivatised dextran polymer and the sterile aqueous buffer solution undergo no detectable degradation at room temperature for at least 12 months after sterilisation.

In an embodiment, the ethylene oxide sterilisation is performed at a temperature of between 30° C. and 60° C. In an embodiment, the ethylene oxide sterilisation is performed at a relative humidity above 30 percent. In an embodiment, the ethylene oxide sterilisation is performed at an ethylene oxide concentration between 200 and 800 mg/l. In an embodiment, the ethylene oxide sterilisation is performed for a duration of at least three hours.

In an embodiment, the sterilisation of the aldehyde-derivatised dextran polymer powder is performed by exposing the aldehyde-derivatised dextran polymer to a dose of between about 25 and about 35 kGy gamma radiation.

In an embodiment, the buffer solution is sterilised by steam sterilisation.

In an embodiment, the relative proportions of N-succinyl chitosan polymer to aldehyde-derivatised dextran polymer present in the kit is between 1:0.2 and 1:1.

In an embodiment, the second container comprises between about 230 and about 440 mg solid aldehyde derivatised dextran polymer.

In an embodiment, the concentration of the N-succinyl chitosan polymer solution is between about 1% and about 10% w/v.

In an embodiment, the first container comprises between about 8 and about 15 ml of N-succinyl chitosan polymer solution.

In an embodiment, the third container comprises between about 8 and about 15 ml of a buffer solution.

In an embodiment, the buffer solution comprises between about 30% w/v and about 50% w/v glycerol.

In an embodiment, the housing further comprises equipment for preparing the hydrogel from the hydrogel precursor components, such as syringes, cannulae and syringe-to-syringe connectors.

In an embodiment, the kit is a single-use kit.

In an embodiment, the method further comprises a step of sterilising the closed housing by ethylene oxide sterilisation.

### Method of Preparation of the Hydrogel

The preparation of the hydrogel from the precursor components may be performed by the combination of the components, allowing sufficient time for the components to crosslink and form a hydrogel.

In an aspect, there is provided a method of preparing a hydrogel comprising combining, in a single container, an aqueous solution with a solid aldehyde-derivatised dextran polymer to prepare an aldehyde-derivatised dextran polymer solution and subsequently adding an aqueous solution comprising N-succinyl chitosan polymer to form a mixture. The mixture is then allowed to form a hydrogel which can be drawn up by a syringe and applied to the site of a surgical wound.

In an aspect, there is provided a method of preparing a hydrogel comprising the steps of:
1. combining an aqueous solution with a solid aldehyde-derivatised dextran polymer to prepare an aldehyde-derivatised dextran polymer solution;
2. combining the aldehyde-derivatised dextran polymer solution with an aqueous solution comprising N-succinyl chitosan polymer to form a mixture;
3. allowing the mixture to form a hydrogel;

wherein the mixture further comprises a humectant.

In an embodiment, the humectant is glycerol.

In an embodiment, step 1 and/or step 2 further comprises agitation of the solution and/or mixture, respectively, to ensure intimate mixing and substantial homogeneity of components throughout the mixture.

In an embodiment, step 1 is performed by adding the contents of the third container (buffer solution) to the second container (aldehyde derivatised dextran polymer), followed by sealing the second container and agitating the contents to form the solution. Agitation can be performed by manually shaking the container for at least 10 seconds, more preferably agitating for 20 seconds. In an embodiment, the solution is drawn up into a first syringe and first cannula. To ensure complete dissolution of the aldehyde derivatised dextran polymer, the solution is allowed to stand for a period of time. Preferably, the solution is allowed to stand for at least 10 minutes, more preferably 15 minutes.

In an embodiment, step 2 is performed by drawing up the N-succinyl chitosan polymer solution using a second syringe and second cannula. The N-succinyl chitosan polymer solution and aldehyde derivatised dextran polymer solution are then combined. In an embodiment, the two solutions are combined by connecting the first and second syringes together via a syringe to syringe connector mixing the solutions by repeatedly transferring the solution between the first and second syringe.

In an embodiment, step 3 is performed by allowing the mixture of N-succinyl chitosan polymer and aldehyde derivatised dextran polymer solution to stand for at least 15 minutes to allow the solution to set into a hydrogel.

In an embodiment, the aqueous solution of step 1 is an aqueous buffer solution. The buffer solution preferably has a pH of between about 7 and 8, more preferably between about 7.2 and 7.6.

In an embodiment, step 2 is performed at least 10 minutes after step 1 is performed. Allowing time between performing step 1 and step 2 ensures that the aldehyde-derivatised dextran polymer has fully dissolved in the aqueous solution.

In an embodiment, the dicarboxy-derivatised chitosan polymer is an N-succinyl chitosan polymer.

In an embodiment, the relative proportions of dicarboxy-derivatised chitosan polymer to aldehyde-derivatised dextran polymer is between 1:0.2 and 1:1, more preferably between 1:0.4 and 1:0.8, most preferably 1:0.6.

In an embodiment, the concentration of the dicarboxy-derivatised chitosan polymer solution is between about 1% and about 10% w/v. In an embodiment, the concentration is between about 3% and about 7% w/v, most preferably about 5% w/v.

In an embodiment, about 8 to 15 ml of aqueous buffer solution is combined with 230 to 440 mg aldehyde derivatised dextran polymer.

In an embodiment, about 8 to 15 ml of aldehyde-derivatised dextran polymer solution is combined with 8 to 15 ml of N-succinyl chitosan polymer solution.

In an embodiment, the components are provided in the correct relative proportions such that the hydrogel may be easily and conveniently prepared from the precursor components.

In an embodiment, the aldehyde-derivatised dextran polymer is provided in an amount that, when the entirety of each component provided in the kit is combined, a hydrogel is prepared having between about 2% to about 8% w/v aldehyde-derivatised dextran polymer, more preferably between about 2% to about 6% w/v aldehyde-derivatised dextran polymer, most preferably about 3% w/v aldehyde-derivatised dextran polymer.

In an embodiment, the dicarboxy-derivatised chitosan polymer solution is provided in an amount that, when the entirety of each component provided in the kit is combined, a hydrogel is prepared having between about 2% to about 8% w/v dicarboxy-derivatised chitosan polymer, more preferably between about 2% to about 6% w/v dicarboxy-derivatised chitosan polymer, most preferably about 5% w/v dicarboxy-derivatised chitosan polymer.

In an embodiment, the hydrogel is suitable for use after about 10 minutes from combining the aldehyde-derivatised dextran polymer solution with the aqueous solution comprising dicarboxy-derivatised chitosan polymer to form a mixture. In an embodiment the hydrogel is suitable for use as a surgical stent or packing material after about 15 minutes. In an embodiment, the surgical wound is related to endoscopic sinus surgery.

In an embodiment, the hydrogel is suitable for use as a surgical stent or packing material for up to 6 hours after its preparation.

Accordingly, in one aspect of the present invention, there is provided a method for preparation of hydrogel comprising the steps of:
1. dissolving a solid aldehyde-derivatised dextran polymer in an aqueous solution to prepare an aldehyde-derivatised dextran polymer solution;
2. combining the aldehyde-derivatised dextran polymer solution with an aqueous solution comprising N-succinyl chitosan polymer to form a mixture;
3. allowing the mixture to form a hydrogel; and wherein the mixture further comprises a humectant.

In an embodiment, the humectant comprises glycerol.

In an embodiment, step 2 is performed at least 10 minutes after step 1 is performed.

In an embodiment, the step of forming a hydrogel takes at least 10 minutes after the aldehyde-derivatised dextran polymer solution and the aqueous solution comprising N-succinyl chitosan polymer are combined.

In an embodiment, 8 to 15 ml of aqueous buffer solution is combined with 230 to 440 mg aldehyde derivatised dextran polymer.

In an embodiment, the concentration of the N-succinyl chitosan polymer solution is between about 1% w/v and about 10% w/v.

In an embodiment, 8 to 15 ml of aldehyde-derivatised dextran polymer solution is combined with 8 to 15 ml of N-succinyl chitosan polymer solution.

In an embodiment, the hydrogel comprises between about 2% to 10% w/v dicarboxy-derivatised chitosan polymer.

In an embodiment, the hydrogel comprises between about 2% to 10% w/v aldehyde-derivatised dextran polymer.

In an embodiment, the hydrogel comprises about 3% w/v aldehyde-derivatised dextran polymer.

### Method of Application of the Hydrogel to a Surgical Wound

In an aspect, there is provided a method for preventing or minimising adhesions associated with a surgical wound in the sinus, comprising preparing the hydrogel of the present invention, and applying the hydrogel to the site of the surgical wound.

In an aspect, there is provided a method for preventing or minimising edema associated with a surgical wound in the sinus, comprising preparing the hydrogel of the present invention, and applying the hydrogel to the site of the surgical wound.

In an aspect, there is provided a method for preventing or minimising granulation associated with a surgical wound in the sinus, comprising preparing the hydrogel of the present invention, and applying the hydrogel to the site of the surgical wound.

In an aspect, there is provided a method of treating a surgical wound comprising the steps of applying a hydrogel comprising aldehyde derivatised dextran polymer, dicarboxy-derivatised chitosan polymer and glycerol to the area of the surgical wound.

In an aspect, there is provided a method of preventing or ameliorating ostial stenosis or reducing ostial patency associated with a surgical wound in the sinus, comprising preparing the hydrogel of the present invention, and applying the hydrogel to the site of the surgical wound.

In an embodiment of the foregoing aspects, the hydrogel is prepared from the kit of the present invention. In an embodiment, the method comprises:
1. dissolving an aqueous solution with a solid aldehyde-derivatised dextran polymer to prepare an aldehyde-derivatised dextran polymer solution;
2. combining the aldehyde-derivatised dextran polymer solution with an aqueous solution comprising N-succinyl chitosan polymer to form a mixture;
3. allowing the mixture to form a hydrogel;

wherein the mixture further comprises a humectant; and

applying the hydrogel to the site of the surgical wound.

In an embodiment of the foregoing aspects, the surgical wound is associated with endoscopic sinus surgery (ESS).

Accordingly, in an aspect of the present invention, there is provided a method of preventing or minimising any one of adhesions, granulation, edema, ostial stenosis or reducing ostial patency associated with a surgical wound in the nasal cavity and/or sinus, comprising:
1. dissolving an aqueous solution with a solid aldehyde-derivatised dextran polymer to prepare an aldehyde-derivatised dextran polymer solution;
2. combining the aldehyde-derivatised dextran polymer solution with an aqueous solution comprising N-succinyl chitosan polymer to form a mixture;
3. allowing the mixture to form a hydrogel;
wherein the mixture further comprises a humectant; and
applying the hydrogel to the site of the surgical wound.

### DETAILED DESCRIPTION

### Definitions

The individual sterilisation procedures described herein would be understood by the skilled person. For the avoidance of doubt, “steam sterilization” typically uses saturated steam at a constant temperature of about 121° C. (250° F.). At this temperature, a holding time of at least 15 minutes is required to achieve sterility.

As used herein the term “chitosan” means a linear polysaccharide composed of randomly distributed B-(1,4) linked D-glucosamine and N-acetyl-D-glucosamine. Chitosan can be produced by deacetylation of chitin. Both α- and β-chitosan are suitable for use in the invention. The degree of deacetylation (% DA) influences the solubility and other properties of the chitosan. Commercially available chitosan typically has a degree of deacetylation of between about 50 to 100%. A monomer unit of fully deacetylated chitosan is shown in formula I below.

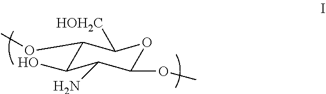

As used herein the term “dicarboxy-derivatised chitosan polymer” means a chitosan polymer that has been derivatised by reaction of a cyclic anhydride with the amine group of some of the D-glucosamine residues of the chitosan polymer. Examples of dicarboxy groups include N-succinyl, N-maloyl and N-phthaloyl. N-succinyl is preferred.

The “dicarboxy-derivatised chitosan polymer” may also be partially derivatised with other functional groups. This secondary derivatisation can occur either at amine positions that are not derivatised with a dicarboxy group or at the hydroxy groups of the D-glucosamine residues. For example, reaction of the cyclic anhydride with an —OH group of the chitosan may lead to some monomers containing ester groups rather than, or in addition to, the amide substituent. If secondary derivatisation is present at the amine position of the dicarboxy-derivatised chitosan polymer, the polymer must retain sufficient free amine groups to be able to form cross-links with the aldehyde-derivatised dextran polymer. Preferably, the dicarboxy-derivatised chitosan polymer is only derivatised by reaction of the cyclic anhydride with the amine group of some of the D-glucosamine residues.

As used herein the terms “N-succinyl chitosan polymer” or “Chitosan N-succinamide” means chitosan that has been derivatised by addition of an N-succinyl group on the amine group of some of the D-glucosamine residues of the chitosan polymer. “N-succinyl chitosan polymer” or “chitosan succinamide” may be used interchangeable. A monomer unit of an N-succinyl chitosan polymer is shown in formula II below.

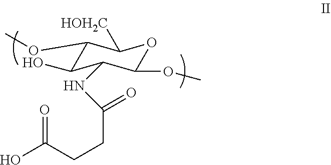

The degree of succinylation may vary. Typically, it is between about 30 to 70%, but the N-succinyl chitosan polymer must retain sufficient free amine groups to be able to form cross-links with the aldehyde-derivatised dextran. The N-succinyl chitosan polymer may also include secondary derivatisation as discussed for the “dicarboxy-derivatised chitosan polymer” (above).

The term “N-succinyl chitosan” as used herein, means an N-succinyl chitosan polymer that is only derivatised with N-succinyl groups at the amine positions and does not include secondary derivatisation with other functional groups.

As used herein the term “dextran” means a glucose polysaccharide composed of α-(1,6) glycosidic linkages with short α-(1,3) side chains. A monomer unit of dextran is shown in formula III below.

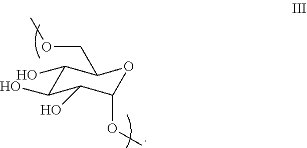

Dextran can be obtained by fermentation of sucrose-containing media by Leuconostoc mesenteroides B512F. Dextrans of molecular weights from 1 KDa to 2000 KDa are commercially available.

As used herein the term “aldehyde-derivatised dextran polymer” means a dextran polymer in which some vicinal secondary alcohol groups have been oxidised to give a reactive bisaldehyde functionality. Aldehyde-derivatised dextran polymers may also be derivatised at other positions with other, functional groups. Preferably, the aldehyde-derivatised dextran polymer is only derivatised at vicinal secondary alcohol groups. A representative monomer unit of aldehyde-derivatised dextran polymer is shown in formula IV below.

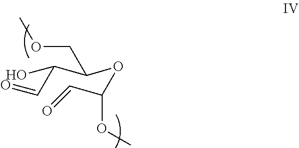

As used herein the term “hydrogel” means a two- or multicomponent system consisting of a three-dimensional network of polymer chains and water that fills the spaces between the macromolecules.

As used herein the term “tissue” means an aggregate of morphologically similar cells with associated intercellular matter that acts together to perform one or more specific functions in the body of an organism including a human. Examples of tissues include but are not limited to muscle, epidermal, nerve and connective tissue.

The term “tissue” also encompasses organs comprising one or more tissue types including but not limited to the chest tissues such as the aorta, the heart, the pleural cavity, the trachea, the lungs, the pericardium and pericardial cavity; the abdominal and retroperitoneal tissues such as the stomach, the small and large intestines, the liver, the pancreas, the gall bladder, the kidneys and the adrenal glands; pelvic cavity tissues including the tissues of the male and female reproductive and urinary tracts; central and peripheral nervous system tissues such as the spinal column and nerves, dura and peripheral nerves; musculoskeletal system tissues such as skeletal muscle, tendons, bones and cartilage; head and neck tissues such as the eye, ear, neck, larynx, nose and paranasal sinuses.

As used herein the term “adhesion” means an abnormal attachment between tissues or organs or between tissues and implants that form after an inflammatory stimulus, such as surgery.

The term “granulation” means the growing of new connective tissue and blood vessels on the surface of a wound.

The term “edema” means the accumulation of extracellular fluid. In the case of edema related to surgery, “edema” means swelling that occurs when too much fluid becomes trapped in the tissues of the body, particularly the skin.

The term “ostial stenosis” means an abnormal narrowing in the blood vessels.

The term “ostial patency” means the degree of openness of a blood vessel and the relative absence of blockage.

Tissues that are susceptible to adhesion formation are tissues that have been exposed to an inflammatory stimulus. For example, tissues which have been involved in surgical procedures such as but not limited to endoscopic sinus surgery, abdominal surgery, gynaecological surgery, musculoskeletal surgery, ophthalmic surgery, orthopaedic surgery and cardiovascular surgery. Tissues may also be susceptible to adhesion formation following other events such as mechanical injury, disease, for example, pelvic inflammatory disease, radiation treatment and the presence of foreign material, for example, a surgical implant.

As used herein the term “wound” means any damage to a tissue in a living organism including human organisms. The tissue may be an internal tissue such as an internal organ or an external tissue such as the skin. The damage may have resulted from a surgical incision or the unintended application of force to the tissue. Wounds include damage caused by mechanical injuries such as abrasions, lacerations, penetrations and the like, as well as burns and chemical injuries. The damage may also have arisen gradually such as occurs in an ulcer, lesion, sore, or infection. Examples of wounds include, but are not limited to, contused wounds, incised wounds, penetrating wounds, perforating wounds, puncture wounds and subcutaneous wounds.

It is intended that reference to a range of numbers disclosed herein (e.g. 1 to 10) also incorporates reference to all related numbers within that range (e.g. 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### The Polymer Network

The invention includes a polymer network formed by derivatisation and cross-linking of two well-known polymers; chitosan and dextran. The polymer rapidly forms a three-dimensional polymer network, creating a hydrogel in aqueous solution. The properties of the hydrogel can be tailored for specific applications by modifying the derivatisation and cross-linking of the two polymer components.

In its broadest aspect the invention provides a polymer network comprising a dicarboxy-derivatised chitosan cross-linked to an aldehyde-derivatised dextran.

The dicarboxy-derivatised chitosan component and aldehyde-derivatised dextran component used in the present invention are described in WO2009/028965, the contents of which are incorporated herein by reference.

### The Chitosan Component

Chitosan is widely available and can be obtained commercially from a range of sources, for example, Sigma-Aldrich (www.sigma-aldrich.com).

Alternatively, chitosan can be prepared by deacetylation of chitin. Many deacetylation methods are known in the art, for example, hydrolysing chitin in a concentrated solution of sodium hydroxide on heating and then recovering chitosan by filtering and washing with water. Chitin exists as either α-chitin or β-chitin. Chitin is found in crustaceans, insets, fungi, algae and yeasts, α-chitin is obtained predominantly from the shells of crustaceans such as lobster, crab and shrimp, whereas β-chitin is derived from squid pens. Both types of chitin can be used to prepare the dicarboxy-derivatised chitosan for use in the invention.

Generally, the average molecular weight (MWₐᵥ) of commercially available chitosan is between about 1 to 1000 kDa. Low molecular weight chitosan has a MWₐᵥ of about 1 to 50 kDa. High molecular weight chitosan has a MWₐᵥ of about 250 to 800 kDa. Chitosan of any MWₐᵥ can be used in the invention.

Deacetylation of chitin means that the resulting chitosan has a majority of free, primary amine groups along its polymeric backbone. The degree of deacylation of the chitosan may influence the properties of the polymer network of the invention because only those glucosamine units that are deacetylated are available for derivatisation or cross-linking. In addition, the solubility of the chitosan depends on the degree of deacylation.

Chitosan polymers most suitable for use in the invention have a degree of deacetylation of between about 40% to 100%. Preferably, the degree of deacylation is between about 60% to 95%, more preferably, between about 70% to 95%.

Chitosans for use in the invention are dicarboxy-derivatised at the amine made free by deacetylation of the chitin. Dicarboxy-derivatised chitosan polymers can be made by reacting chitosan with a cyclic acid anhydride. Cyclic acid anhydrides suitable for use in the invention include succinic anhydride, maleic anhydride, phthalic anhydride, glutaric anhydride, citraconic anhydride, methylglutaconic anhydride, methylsuccinic anhydride and the like.

Preferably, the dicarboxy-derivatised chitosan polymer is made from the reaction of chitosan and one or more of succinyl anhydride, phthalic anhydride, or glutaric anhydride. More preferably, the dicarboxy-derivatised chitosan polymer is made from the reaction of chitosan and succinyl anhydride.

Derivatisation can be achieved by any method known in the art. For example, the solid chitosan can be heated in a solution of cyclic anhydride in DMF or solubilised in a methanol/water mixture and then reacted with the anhydride. Other solvents suitable for use in the derivatisation process include dimethylacetamide. Acids such as lactic acid, HCl or acetic acid can be added to improve the solubility of the chitosan. A base such as NaOH is typically added to deacelylate some of the acetylated amine groups.

An exemplary method is provided in WO2009/028965. The method used can be selected depending on the cyclic anhydride used and/or the average molecular weight of the chitosan. Both the chitosan and the cyclic anhydride should be able to substantially dissolve or swell in the solvent used.

In a preferred embodiment, the dicarboxy-derivatised chitosan is N-succinyl chitosan. Methods of preparing N-succinyl chitosan are well known in the art. See for example, “Preparation of N-succinyl chitosan and their physical-chemical properties”, J Pharm Pharmacol. 2006, 58, 1177-1181.

The reaction of the cyclic anhydride with the chitosan acylates some of the free amine positions with dicarboxy groups. For example, when the cyclic anhydride used is succinic anhydride, some of the amine groups are N-succinylated. The NaOH treatment following N-succinylation removes some of the acyl groups from the amine groups in the chitosan. Increasing the temperature of the NaOH treatment increases the percentage of free amine groups present, as demonstrated in WO2009/028965.

The degree of acylation is indicated by the ratio of C:N in the product. The degree of acylation can also be determined by ¹H nmr. An N-succinyl chitosan polymer is represented below. Formula V shows the three types of D-glucosamine units present in the polymer-the N-succinylated-D-glucosamine, the free D-glucosamine, and the N-acetyl-D-glucosamine.

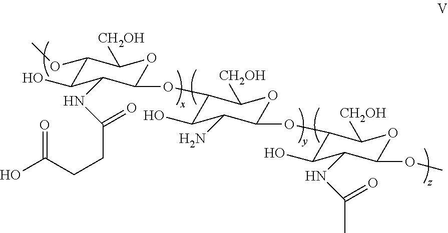

In one embodiment, x is between about 60 to 80%, y is between about 1 to 15% and z is between about 10 to 25%.

In another embodiment, x is between about 60 to 80%, y is between about 1 to 30% and z and between about 2 to 25%.

High degrees of anhydride substitution render the dicarboxy-derivatised chitosan polymer more soluble but may hinder cross-linking to the aldehyde-derivatised dextran polymer.

In one embodiment, the dicarboxy-derivatised chitosan polymer is between about 20% and 80% dicarboxy derivatised. Preferably, the dicarboxy-derivatised chitosan polymer is between about 30% and 60% dicarboxy derivatised. More preferably, dicarboxy-derivatised chitosan polymer is between about 45% and 50% dicarboxy derivatised. In one embodiment, the dicarboxy-derivatised chitosan polymer is between about 50% and 90% dicarboxy derivatised. Preferably, the dicarboxy-derivatised chitosan polymer is between about 60% and 80% dicarboxy derivatised.

### The Dextran Component

Dextran is a polysaccharide made of D-glucose units linked predominantly by α-1,6 linkages. Crude, high molecular weight dextran is commercially obtained by growing Leuconostoc mesenteroies on sucrose. The resulting polysaccharide is hydrolysed to yield low molecular weight dextrans.

Before dextran can be cross-linked to the dicarboxy-derivatised chitosan polymer, it must be activated. Reactive bisaldehyde functionalities can be generated from the vicinal secondary alcohol groups on dextran by oxidation. Typical methods are provided in WO2009/028965. The resulting aldehyde-derivatised dextran polymer can then be reductively coupled to the primary amine groups of the dicarboxy-derivatised chitosan to form a cross-linked polymer network of the invention.

In one embodiment, the oxidising agent is sodium periodate. Other suitable oxidising agents include potassium periodate and the like.

The oxidised product, the aldehyde-derivatised dextran polymer, actually only contains a small amount of free aldehyde groups. Most of the aldehyde groups are masked as acetals and hemiacetals, which are in equilibrium with the free aldehyde form of the dextran. Reaction of some of the free aldehyde groups causes the equilibrium to shift from the acetal and hemiacetal form, towards the formation of more free aldehyde groups.

The degree of oxidation can be influenced by the molar ratio of oxidising agent used. A higher degree of oxidation will provide an aldehyde-derivatised dextran polymer with more sites available for cross-linking. However, a lower degree of oxidation will result in a more soluble aldehyde-derivatised dextran polymer. The periodate reaction also dramatically decreases the molecular weight of the dextran polymer.

In one embodiment, the degree of oxidation is between about 30% to about 100%, more preferably between about 50% to about 100%. Most preferably, the degree of oxidation is between about 80 to about 100%. WO2009/028965 compares gelling times for polymer networks of the invention prepared using aldehyde-derivatised dextran polymers with different degrees of aldehyde-derivatisation (or oxidation). More highly aldehyde-derivatised dextran polymers have lower molecular weights and form gels faster, when combined in solution with solutions of N-succinyl chitosan.

The degree of derivatisation can be measured using the extended reaction with hydroxylamine hydrochloride and then titration of the liberated protons (Zhao, Huiru, Heindel, Ned D, “Determination of degree of substitution of formyl groups in polyaldehyde dextran by the hydroxylamine hydrochloride method,” Pharmaceutical Research (1991), 8, page 400-401).

It has been found that the aldehyde groups of the aldehyde derivatised dextran polymer are susceptible to react with nearby hydroxyl groups, forming hemiacetals or hemialdals, in water. Aldehyde derivatised dextran polymer will therefore degrade over time in an aqueous solution, causing to shorter viable shelf life. Therefore, it has been found that the aldehyde derivatised dextran polymer should be stored as a solid to maximise its shelf life, particularly beyond 12 months from manufacture.

Cross-Linking the Chitosan Component with the Dextran Component

The invention provides a polymer network comprising a dicarboxy-derivatised chitosan polymer cross-linked to an aldehyde-derivatised dextran polymer. In one embodiment the dicarboxy-derivated chitosan polymer is an N-succinyl chitosan polymer. In one embodiment the N-succinyl chitosan polymer is cross-linked to the aldehyde-derivatised dextran polymer through the amine group of the N-succinyl chitosan polymer and the aldehyde group of the aldehyde-derivatised dextran polymer. Preferably, the N-succinyl chitosan polymer is N-succinyl chitosan.

The invention also provides a method of producing a polymer network as described above.

To make a polymer network of the invention, the dicarboxy-derivatised chitosan polymer is cross-linked to the aldehyde-derivatised dextran polymer. This can be achieved by mixing aqueous solutions of the two polymers. For example, see WO2009/028965.

In one embodiment, it is desirable that the aqueous solution in which the polymer matrix forms has a pH of about 6 to 8, preferably between about 6.5 to 7.5. This can be achieved by adjusting the pH of the separate aqueous solutions of the polymer components to within this range before mixing the two solutions. Alternatively, the pH of the aqueous solutions of the individual polymer components can be adjusted following dialysis, prior to freeze drying. The pH can be adjusted using any suitable base or acid. Generally, the pH will be adjusted using NaOH.

In one embodiment either or both of the aqueous solutions may independently contain one or more pharmaceutically acceptable excipients. In one embodiment the aqueous solutions may independently contain NaCl. Preferably, the concentration of NaCl is between about 0.5 to 5% w/v. More preferably, the concentration of NaCl is between about 0.5% to 2% w/v, most preferably about 0.9% w/v.

In one embodiment the aqueous solutions may independently contain one or more buffers including but not limited to phosphate buffers such as sodium phosphate (e.g. Na₂HPO₄), acetate buffers, carbonate buffers, lactate buffers, citrate buffers and bicarbonate buffers.

### The Humectant Component

The presence of a humectant helps in keeping the gel soft without affecting the crosslinking properties or gel structure. In a particular example, the hydrogel comprises about 20% glycerol. The use of a humectant allows the hydrogel to be prepared longer in advance of its use because the humectant reduces the loss of moisture to the environment and thus maintains the desired physical characteristics of the hydrogel for a longer period of time. The hydrogel may be prepared prior to the surgery and can be used within 6 hours after preparation, if the surgery lasts longer than usual time of 2-3 h. The hydrogel does not lose its physical or chemical properties within the 6 hour time frame. In an embodiment, the humectant is glycerol.

### The Hydrogels of the Invention

The dicarboxy-derivatised chitosan polymer reacts with the aldehyde-derivative dextran polymer, to produce a three-dimensional cross-linked polymer network. This polymer network forms a hydrogel with the aqueous solution in which it is formed. The hydrogel of the invention has properties that make it suitable for use in medicinal applications, in particular, wound healing, prevention of surgical adhesions, and reducing bleeding (haemostasis).

Without being bound by theory, it is believed that application of the hydrogel of the invention to a wound surface prevents the formation of fibrin and blood clots within this space thereby preventing subsequent formation of adhesions.

The properties of the hydrogel can be tailored for specific applications by modifying the derivatisation and cross-linking of the two polymers.

In the polymer networks of the invention, the amine groups of the D-glucosamine residues of chitosan may be

(a) cross-linked to the aldehyde-derivatised dextran polymer,

(b) acylated with a dicarboxy group, or

(c) acetylated (from the original chitin material).

High degrees of acetylation and/or dicarboxy acylation will leave less free amine groups to cross-link with the aldehyde-derivatised dextran polymer. Consequently, when the aqueous solutions of the two polymers are mixed, the amount of polymerisation that occurs will be affected by the acylation and acetylation patterns of the dicarboxy-derivatised chitosan polymer. This in turn will affect how quickly, if at all, the hydrogel is formed. If very little polymerisation occurs in a dilute solution of the polymers, no hydrogel will be formed.

The aqueous solutions of dicarboxy-derivatised chitosan polymer and aldehyde-derivatised dextran polymer comprise between about 2% to about 10% w/v of each component.

Generally, aqueous solutions of equal concentrations of the two polymers are mixed to form the hydrogel of the invention. However, different ratios of dicarboxy-derivatised chitosan polymer and aldehyde-derivatised dextran polymer can be used, provided the properties of the two polymers are such that they cross-link to form a hydrogel of the invention when mixed together.

A person skilled in the art can manipulate the parameters of

(a) degree of deacetylation of chitosan,

(b) degree of dicarboxy-derivatisation of chitosan,

(c) degree of oxidation of aldehyde-derivatised dextran, and

(d) concentration in aqueous solution,

so that the component polymer solutions rapidly cross-link to form a hydrogel when mixed.

### Kits

A kit of the present invention comprises separately sealed hydrogel precursors, equipment for preparing the hydrogel, instructions for preparing the hydrogel and a sterile barrier system for housing the components of the kit. An exemplary kit comprises hydrogel precursors:
Sealed vial A containing 12 ml Sodium Phosphate buffer solution containing 40% w/v glycerol
Sealed vial B containing 350 mg Aldehyde-derivatised dextran polymer powder
Sealed vial C containing 12 ml N-succinyl chitosan polymer (5% w/v) solution in 0.3% sodium phosphate buffer.

The exemplary kit further comprises equipment for preparing the hydrogel:
Fluid dispensing connector
Two (2) mixing cannulae
Pliable cannula
Two (2) sterile 20 ml Luer Lock syringes
Barrier system for containing the precursors and equipment

Once the barrier system has been sterilised by ethylene oxide sterilisation treatment, all components of the kit are sterile and suitable for use in an operating theatre.

A non-sterile protective packaging may be used to enclose the sterile barrier system in order to prevent damage to the sterile barrier system and its contents

The kits of the invention may be further adapted to comprise one or more biologically active agents. For example, the one or more biologically active agents can be combined with one or both of the dicarboxy-derivatised chitosan and aldehyde derivatised dextran.

As noted above, the aldehyde derivatised dextran polymer is provided as a solid to ensure its stability over a shelf life of at least 12 months. The aldehyde derivatised dextran polymer may advantageously be in an amorphous form.

Whilst N-succinyl chitosan polymer could be provided as a solid, and dissolved into a solution by an end-user of the kit, it has been found by the inventors that N-succinyl chitosan polymer is advantageously provided in the kit as a solution, as opposed to a solid form. This is because N-succinyl chitosan polymer is hygroscopic, and it is therefore easier to handle during the manufacturing process as a solution. Further, N-succinyl chitosan polymer has been found to be slow to dissolve in aqueous solution. For convenience in manufacturing, as well as in the preparation of the hydrogel, the N-succinyl chitosan polymer is thus provided as a solution.

The pliable cannula comprises a soft plastic tube and two stainless steel pliable wires embedded in the plastic tube. The wires are entirely surrounded by the plastic tubing, and extend substantially along the whole length of the plastic tube. A first open end of the pliable cannula is configured to be attached to a syringe, for example via a luer lock or luer slip mechanism and a second open end of the pliable cannula is configured to apply the hydrogel to the site of the surgical wound.

### Method of Sterilisation of the Kit

The contents of the kit, including the kit housing, are sterile and suitable for use in an operating theatre.

### Aldehyde-Derivatised Dextran Polymer

The most sensitive precursor component is aldehyde-derivatised dextran polymer. Aldehyde groups are susceptible to react with nearby hydroxyl groups, forming hemiacetals or hemialdals, in water. Thus, steam sterilisation is not possible for the dry dextran powder.

Due to presence of aldehyde groups, the inventors believe that UV irradiation may produce free radicals in solid state aldehyde-derivatised dextran polymer, leading to an irreversibly crosslinked aldehyde-derivatised dextran polymer product. Hence, neither steam nor UV irradiation is suitable for the sterilization of aldehyde-derivatised dextran polymer.

Gamma irradiation is known to generate free radicals in a dose dependent manner. Free radicals are capable of cleaving glycosidic bonds leading to depolymerization of polysaccharides. The inventors have found that, in the case of aldehyde-derivatised dextran polymer, gamma irradiation-induced free radicals only cleave the glycosidic bonds when the polymer is in solution. Further surprisingly, gamma irradiation was found not to affect the relative frequencies of the different linkages in aldehyde-derivatised dextran polymer in the solid phase. That is, it has surprisingly been found that gamma irradiation does not substantially degrade aldehyde-derivatised dextran polymer. Thus, gamma irradiation has been found by the inventors to be a suitable sterilization method for aldehyde-derivatised dextran polymer.

Gamma sterilization has an additional advantage of a high penetrating ability, relatively low chemical reactivity and instantaneous results without need to control the temperature, pressure, vacuum or humidity.

Aldehyde-derivatised dextran polymer was exposed to gamma radiation from a Cobalt 60 radiation source. A dose of at least 25 kGy to the aldehyde-derivatised dextran polymer was found to achieve a Sterility Assurance Level (SAL) of 10⁻⁶ during validation of sterilization processes.

After the exposure to gamma irradiation, the aldehyde-derivatised dextran polymer displayed no major change in solubility time or gelation properties.

### Buffer Sterilization

The buffer solution of the kit comprises an aqueous solution of sodium phosphate. The buffer solution contains no pharmaceutically or biologically active ingredients or ingredients that are susceptible to degradation by sterilisation procedures involving heat, steam or gamma radiation. Thus, the buffer solution was found to be able to be sterilised by gamma radiation, steam sterilisation or heat sterilisation.

### N-Succinyl Chitosan Polymer Sterilization

The inventors have found that exposure of N-succinyl chitosan polymer to gamma or beta ionizing radiation induces degradation of the polymer through chain scission. Further, the inventors believe that UV irradiation may lead to formation of free radicals and destruction of polymer amino groups in N-succinyl chitosan polymer.

Without wishing to be bound by theory, the inventors believe that the rate of degradation in highly deacetylated chitosan (˜85% deacetylated chitosan) is higher under radiative sterilisation procedures compared to acetylated chitosan or less deacetylated chitosan, which in turn affects the viscosity of N-succinyl chitosan polymer solutions and negatively affects the hydrogel formation when combined with aldehyde-derivatised dextran polymer.

Exposure of N-succinyl chitosan polymer to ethylene oxide sterilisation procedures was found to not produce any degradation of the polymer, but this sterilization technique has limited use for chitosan-based materials in dry state. This is because N-succinyl chitosan is hygroscopic and is therefore prone to absorb moisture during the ethylene oxide sterilisation process.

Surprisingly, the inventors have found saturated water steam sterilization to be an appropriate method for the sterilisation of chitosan solutions and N-succinyl chitosan polymer solutions. The inventors have found that a N-succinyl chitosan polymer solution, having a pH range between 6 and 8, does not undergo any appreciable degradation during saturated steam sterilisation procedures.

It has been found that steam sterilization treatment does not alter the chemical structure of the N-succinyl chitosan polymer. It has been further found that that steam sterilisation treatment does not affect the gelation time of a hydrogel formed between N-succinyl chitosan polymer and aldehyde-derivatised dextran polymer.

### Sterilization of the Kit, Equipment and Barrier System

The sterilised N-succinyl chitosan polymer solution, sterilised buffer solution and sterilised aldehyde-derivatised dextran polymer were each transferred to separate glass vials and sealed with resin caps.

The kit comprises two 20 ml syringes, three glass vials comprising the N-succinyl chitosan, aldehyde derivatised dextran and buffer solution, two mixing cannulae, a pliable cannula, a syringe to syringe connector, and a barrier system for housing the precursors and equipment. A cardboard package having the product labelling is also provided, which is adapted to receive the barrier system.

In an alternative embodiment, the kit further comprises a foam inner, a hard plastic shell and/or product labelling. The foam inner comprises indentations for receiving each component.

Each vial comprising a hydrogel precursor component was placed into the kit barrier system. The barrier system is adapted to be able to be sealed closed and maintain a sterile seal during closure. The barrier system was loaded with the capped and sealed glass vials of each hydrogel precursor component. The barrier system was further loaded with a fluid dispensing connector, two mixing cannulae, a pliable cannula, two sterile 20 ml Luer lock syringes. The barrier system, in an open configuration, was then exposed to an ethylene oxide sterilisation procedure.

As the kit is sterilised by ethylene oxide, the vials, lids, equipment, barrier system and labelling must not be susceptible to degradation by ethylene oxide or any of the required process parameters (such as, the temperatures, humidities and pressures of the ethylene oxide sterilisation process).

The development of the sterilisation procedure for the preparation for the kit has overcome several significant challenges. The inventors have found that the aldehyde derivatised dextran/N-succinyl chitosan hydrogel suffers from instability in consistency over the course of days or weeks. Accordingly, the inventors set out to prepare a kit comprising the hydrogel precursors which could be used by health care workers to prepare the hydrogel at the point of use. Significant challenges in the development of a sterile kit were how to sterilise each precursor, the precursor packaging and the entire kit in such a way that none of the components were damaged or degraded during the sterilisation procedure. For example, the inventors have found that a combination of solid aldehyde derivatised dextran and solid N-succinyl chitosan (e.g. combined in a single container) could not be sterilised by any method of steam sterilisation (as N-succinyl chitosan is hygroscopic), gamma radiation (as N-succinyl chitosan is degraded by gamma radiation) or ethylene oxide (as N-succinyl chitosan is hygroscopic). In addition, the inventors have found that aldehyde derivatised dextran could not be provided as an aqueous solution, because it would degrade under gamma radiation in solution (but not as a solid).

### Preparation of a Hydrogel Stent or Packing Material

11 ml of the contents of sealed vial A (sodium phosphate buffer solution) was drawn up by a first sterile 20 ml Luer lock syringe and transferred to opened vial B. Vial B was capped and agitated for 20 seconds. The AB mixture was drawn up into the first syringe and allowed to stand for 15 minutes to ensure dissolution of the aldehyde derivatised dextran polymer powder.

11 ml of sealed vial C was drawn up into a second sterile 20 ml Luer lock syringe. The first and second syringes were connected together via a syringe to syringe connector and the AB mixture and C mixture were combined and mixed by transferring the solution between the first and second syringe at least 6 times. The ABC solution was allowed to stand for at least 15 minutes to allow the solution to set into a hydrogel.

The concentration and relative proportion of each component has been determined by the inventors to allow an easy and convenient preparation procedure for health workers. For example, if the concentrations of the AB mixture and the C mixture are too high, or the relative proportion of aldehyde derivatised dextran polymer is too high relative to N-succinyl chitosan, the formation of a hydrogel can be too rapid (resulting in a hydrogel mixture which is not sufficiently homogenous), or the consistency of the hydrogel will be too hard. Conversely, if the concentrations are too low, then the hydrogel will take too long to form a suitably viscous consistency.

### Treatment of Surgical Wounds

The hydrogel, prepared according to the method described above, is useful as a stent or packing material. In particular, the hydrogel is a packing material. The hydrogel acts as a space-occupying packing material for use as a stent or packing material for application to surgical wounds, particular surgical wounds associated with ESS.

The hydrogel forms in situ in two 20 ml syringes following the combination of components A, B and C. Once it has formed a gel, it may be applied to the site of a surgical wound. For surgical wounds associated with ESS, the pliable cannula is particularly advantageous in delivering the hydrogel safely and effectively to the wound.

The pliable wires embedded in the tube allow the pliable cannula to be manually bent and shaped into a particular configuration so as to allow the cannula to be inserted via the nose (for example) or other body orifice to the site of the surgical wound. The pliability of the cannula thus allows the precise application of the hydrogel to the site of the surgical wound without causing further trauma to the patient.

To apply the hydrogel to a surgical wound associated with ESS, the pliable cannula is attached to the end of the syringe containing the hydrogel. The cannula is manually shaped by the health care worker to allow the cannula to be inserted through the nostril to the site of the surgical wound such that the second opening of the cannula is oriented towards the site of the wound. A layer of the hydrogel is then applied to the wound.

The plastic tube itself is a soft plastic material which is sufficiently soft that it does not cause trauma to the patient during insertion of the cannula or application of the hydrogel.

The invention provides the hydrogel to:
Separate tissue or structures compromised by surgical trauma;
Separate and prevent adhesions between mucosal surfaces in the nasal cavity and minimize ostial stenosis following endoscopic sinus surgery;
Control minimal bleeding following surgery or trauma by tamponade effect, blood absorption, and platelet aggregation; and
Act as an adjunct to aid in the natural healing process.

The hydrogel of the present invention is prepared and applied to a surgical wound associated with ESS. Application of the hydrogel to surgical wounds associated with ESS has been found to reduce incidences of adhesions, granulation and edema associated compared to non-hydrogel packing material or no packing material.

Compared to using no packing materials at 12 months post surgery, patients receiving the hydrogel of the present invention on average experienced the following:
73% improvement in frontal ostial area
35% improvement in sphenoid ostial area
34% improvement in maxillary ostial area
47% reduction in incidence of adhesion
50% reduction in incidence of edema
50% reduction in incidence of granulation

1. A kit for preparing a hydrogel comprising:
a first container comprising an aqueous solution comprising N-succinyl chitosan polymer;
a second container comprising a solid aldehyde-derivatised dextran polymer;
a third container comprising an aqueous buffer solution for suspending the aldehyde-derivatised dextran polymer during the preparation of the hydrogel; and
a sterilisable barrier system enclosing the first, second and third containers;
wherein a humectant is present in either or both of the first and third containers.

2. The kit of claim 1, wherein the humectant is glycerol.

3. The kit of claim 1, wherein the sterilisable barrier system is permeable to ethylene oxide.

4. The kit of claim 1, wherein the kit further comprises equipment for preparing the hydrogel, such as syringes, cannulae and syringe to syringe connectors.

5. The kit of claim 4, wherein the equipment includes a pliable cannula comprising a soft plastic tube and one or more pliable wires embedded in the plastic tube, wherein the pliable wire(s) extends partially or completely along the length of the plastic tube.

6. The kit of claim 5, wherein the pliable cannula comprises a first open end configured to be attached to a syringe and a second open end of the pliable cannula configured to apply the hydrogel to the site of a surgical wound associated with endoscopic sinus surgery.

7. The kit of claim 1, wherein the N-succinyl chitosan is suspended in water, saline, or a buffer solution, such as a sodium phosphate buffer.

8. The kit of claim 1, wherein the aldehyde-derivatised dextran polymer is provided as a dry solid.

9. The kit of claim 1, wherein the concentration of the N-succinyl chitosan polymer solution is between about 1% and about 10% w/v.

10. The kit of claim 1, wherein the relative proportions of N-succinyl chitosan polymer to aldehyde-derivatised dextran polymer present in the kit is between 1:0.2 and 1.1.

11. The kit of claim 1, wherein the second container comprises between about 230 and about 440 mg solid aldehyde derivatised dextran polymer.

12. The kit of claim 1, wherein the concentration of the N-succinyl chitosan polymer solution is between about 1% and about 10% w/v.

13. The kit of claim 12, wherein the first container comprises between about 8 and about 15 ml of N-succinyl chitosan polymer solution.

14. The kit of claim 1, wherein the third container comprises between about 8 and about 15 ml of a buffer solution.

15. The kit of claim 14, wherein the buffer solution comprises between about 30% w/v and about 50% w/v humectant.

16. A method for sterilising a kit for preparing a sterile hydrogel, the method comprising the steps of:
i. sealing a solution of N-succinyl chitosan polymer in a first container;
ii. sterilising the sealed first container comprising the N-succinyl chitosan polymer by steam sterilisation;
iii. sealing a solid aldehyde-derivatised dextran polymer in a second container;
iv. sterilising the second container comprising the solid aldehyde-derivatised dextran polymer by gamma irradiation sterilisation;
v. sealing an aqueous buffer solution in a third container;
vi. sterilising the third container comprising the aqueous solution;
vii. sealing the first, second and third sterile sealed containers in a housing;
viii. sterilising the housing comprising the first, second and third containers by exposure to ethylene oxide.

17. The method of claim 16, wherein the sterile aqueous solution comprising N-succinyl chitosan polymer, the sterile solid aldehyde-derivatised dextran polymer and the sterile aqueous buffer solution undergo no detectable degradation at room temperature for at least 12 months after sterilisation.

18. The method of claim 16, wherein the solid aldehyde-derivatised dextran polymer is a powder.

19. The method of claim 16, wherein the buffer solution comprises between about 30% w/v and about 50% w/v glycerol.

20. A method for preparation of a hydrogel comprising the steps of:
1. dissolving a solid aldehyde-derivatised dextran polymer in an aqueous solution to prepare an aldehyde-derivatised dextran polymer solution;
2. combining the aldehyde-derivatised dextran polymer solution with an aqueous solution comprising N-succinyl chitosan polymer to form a mixture;
3. allowing the mixture to form a hydrogel; and
wherein the mixture further comprises a humectant.

21. The method of claim 20, wherein the humectant is glycerol.

22. The method of claim 20, wherein step 2 is performed at least 10 minutes after step 1 is performed.

23. The method of claim 20, wherein the step of forming a hydrogel takes at least 10 minutes after the aldehyde-derivatised dextran polymer solution and the aqueous solution comprising N-succinyl chitosan polymer are combined.

24. The method of claim 20, wherein 8 to 15 ml of aqueous buffer solution is combined with 230 to 440 mg aldehyde derivatised dextran polymer.

25. The method of claim 20, wherein the concentration of the N-succinyl chitosan polymer solution is between about 1% w/v and about 10% w/v.

26. The method of claim 20, wherein 8 to 15 ml of aldehyde-derivatised dextran polymer solution is combined with 8 to 15 ml of N-succinyl chitosan polymer solution.

27. The method of claim 20, wherein the hydrogel comprises between about 2% to 10% w/v dicarboxy-derivatised chitosan polymer.

28. The method of claim 20, wherein the hydrogel comprises between about 2% to 10% w/v aldehyde-derivatised dextran polymer.

29. The method of claim 20, wherein the hydrogel comprises about 3% w/v aldehyde-derivatised dextran polymer.

30. Method of preventing or minimising any one of adhesions, granulation, edema, ostial stenosis or reducing ostial patency associated with a surgical wound in the nasal cavity and/or sinus, comprising:
1. dissolving a solid aldehyde-derivatised dextran polymer in an aqueous solution to prepare an aldehyde-derivatised dextran polymer solution;
2. combining the aldehyde-derivatised dextran polymer solution with an aqueous solution comprising N-succinyl chitosan polymer to form a mixture;
3. allowing the mixture to form a hydrogel;
wherein the mixture further comprises a humectant; and
applying the hydrogel to the site of the surgical wound in the nasal cavity and/or sinus.
